(19) United States
(12) Patent Application Publication
(10) Pub. No.: US 2014/0272954 A1
(43) Pub. Date: Sep. 18, 2014

(54) METHODS AND SYSTEMS FOR ELECTRONIC KARYOTYPING

(71) Applicant: NABsys, Inc., Providence, RI (US)

(72) Inventors: Stan Rose, Falmouth, MA (US); Ulrich Broeckel, Wauwatosa, WI (US)

(73) Assignee: NABsys, Inc., Providence, RI (US)

(21) Appl. No.: 14/200,601

(22) Filed: Mar. 7, 2014

Related U.S. Application Data
(60) Provisional application No. 61/787,138, filed on Mar. 15, 2013.

(51) Int. Cl. C12Q 1/68

(52) U.S. Cl. CPC C12Q 1/6816
USPC 435/6.11

(57) ABSTRACT

Embodiments of the present invention relate to a method for producing patterns of sequence specific markers on a chromosomal segment. By comparing these patterns to those produced on a reference chromosome, various genetic abnormalities can be detected.

### CROSS REFERENCE TO RELATED APPLICATION

This application claims the benefit of priority under 35 U.S.C. §119(e) to U.S. Provisional Patent Application Ser. No. 61/787,138 filed Mar. 15, 2013, which is incorporated herein by reference in its entirety.

### BACKGROUND

Cytogenetics is the study of chromosome structures and genomic aberrations that cause disease. The number and appearance of chromosomes have traditionally been analyzed by karyotyping, a method of staining chromosomes with dye to create patterns of light and dark bands that can be seen under a fluorescent microscope. Although karyotyping is considered the gold standard in cytogenetics, the technology tends to be time-consuming, expensive, subjective, and has poor resolution.

Fluorescent In Situ Hybridization (FISH) was developed as an adaptation of classical cytogenetics that uses biomolecule probes to analyze specific chromosomal events. See, e.g., Langer-Safer P R, et al., (July 1982), “Immunological method for mapping genes on Drosophila polytene chromosomes,” Proc. Natl. Acad. Sci. U.S.A. 79 (14): 4381-5, and U.S. Pat. No. 8,278,050 to Bailey et al.; both publications are incorporated herein by reference in their entireties. Compared to karyotyping, FISH has higher resolution and shorter turnaround times; however, important information about the entire genome is missed since only biomolecule locations are probed.

More recently, cytogeneticists have been replacing conventional karyotyping and FISH panels with microarray-based diagnostics that use DNA probes bound to glass slides to analyze chromosomes. See, e.g., U.S. Pat. No. 8,232,055 to Bruhm et al., incorporated herein by reference in its entirety. Array-based comparative genomic hybridization (aCGH) technology offers a semi-automated test for genome wide screening, with resolution and specific regions covered dependent on the number and nature of the probes on the array. Several disadvantages of aCGH are its inability to analyze all types of genomic variations (e .g., balanced translocations) and its relatively low resolution due to the limited number, length and spacing of probes on the array.

### SUMMARY

In one aspect, embodiments of the invention relate to a method for analyzing chromosomal structures. More specifically, the method includes the steps of providing a chromosomal segment to be analyzed, and providing at least one sequence-specific marker set, such that each marker within the set binds or otherwise locates at a sequence-specific location on the segment to be analyzed. The marker sets are then allowed to bind or otherwise locate on the chromosomal segment to form an analyte. The analyte is introduced into a device having a nanodetector and the analyte is translocated through the nanodetector. During the translocation, electrical properties at the nanodetector are monitored, and changes indicative of i) no chromosomal segment in the nanodetector, ii) a portion of the chromosomal segment lacking a marker present in the nanodetector and iii) a portion of the chromosomal segment having a marker present in the nanodetector are detected and recorded. When the changes indicative of the presence of a marker on the chromosomal segment are recorded relative to their positions on the segment, a distinctive pattern is formed. That pattern may be compared to an analogous pattern on a reference chromosome to detect and determine abnormalities in the chromosomal segment being analyzed.

In another aspect, embodiments of the invention relate to a method for analyzing structural variation in genomes. The method includes the steps of isolating DNA representing an entire genome or a portion thereof to be analyzed, providing at least one sequence-specific marker set, with each marker in the set capable of binding to or otherwise locating at a specific sequence that appears one or more times in the isolated DNA, and allowing the one or more marker sets to bind or otherwise locate on the isolated DNA to form an analyte. The analyte is then introduced into a device having a nanodetector including a detection volume, and translocated through the detection volume. During the translocation, electrical properties at the detection volume are detected and monitored, and changes indicative of i) no DNA segment in the detection volume, ii) a portion of the DNA segment lacking a marker present in the detection volume, and iii) a portion of the DNA segment having a marker present in the detection volume are detected and recorded. The changes in the electrical properties relative to their positions on the analyte are recorded to reveal a pattern of changes in the electrical signal that is related to the positions at which markers are present on the isolated DNA. This pattern may then be compared to analogous patterns from control and test samples to reveal differences that are indicative of specific abnormalities or structural variations in the test sample.

Embodiments of the invention in accordance with each of these aspects may include one or more of the following features. At least a portion of the analyte may be coated with a binding moiety, preferably being one or more proteins, and more preferably being at least a protein selected from among RecA, T4 gene 32 protein, f1 geneV protein, human replication protein A, Pf3 single-stranded binding protein, adenovirus DNA binding protein, and E. coli single-stranded binding protein. The nanodetectors may take the form of fluidic nanopores, fluidic micro-channels or fluidic nano-channels. Markers may be oligonucleotide probes, and they may include tags which enhance the ability of the marker to be detected by the nanodetector. The pattern distinctive of the chromosomal segment may be quantified.

### BRIEF DESCRIPTION OF DRAWINGS

FIG. 1 is a representation of a conventional karyogram, showing its relationship to a simulated karyogram developed using the electronic karyotyping methods of embodiments of the present invention.

FIG. 2a is a schematic representation of a nanodetector employing a nanopore for electronic karyotyping in accordance with an embodiment of the invention.

FIG. 2b is an exemplary waveform obtained with the nanodetector of FIG. 2a.

FIG. 3 is a schematic representation of a nanodetector employing a fluidic nano-channel or micro-channel for electronic karyotyping.

FIGS. 4a-4e are schematic representations of one embodiment of a method for electronic karyotyping in a nanodetector employing a fluidic nano-channel or micro-channel, in accordance with an embodiment of the invention.

FIG. 5 is a schematic representation of a normal chromosomal fragment, analyzed by the methods in accordance with embodiments of the present invention and compared against a reference region of a chromosome.

FIG. 6 is a schematic representation of a chromosomal fragment having a deletion, analyzed by the methods in accordance with embodiments of the present invention and compared against a reference region of a chromosome.

FIG. 7 is a schematic representation of a chromosomal fragment having a duplication, analyzed by the methods in accordance with embodiments of the present invention and compared against a reference region of a chromosome.

FIG. 8 is a schematic representation of a chromosomal fragment having a translocation, analyzed by the methods in accordance with embodiments of the present invention and compared against a reference region of a chromosome.

FIG. 9 is a schematic representation of a chromosomal fragment having an inversion, analyzed by the methods in accordance with embodiments of the present invention and compared against a reference region of a chromosome.

FIG. 10a is a data trace of a segment of DNA from Saccharomyces cerevisiae obtained using the methods in accordance with embodiments of the present invention.

FIG. 10b is a pattern representative of the data trace of FIG. 10a.

### DETAILED DESCRIPTION

Chromosomal disorders are relatively common in humans. They are found in about 1% of all live births, 2% of pregnancies in women over 35, and approximately 7% of autism related disorders. Most cancers have acquired structural or numerical chromosomal abnormalities. The consequences can vary between those affected. Some chromosomal abnormalities do not affect the health, while other chromosomal abnormalities are associated with clinical manifestations and therefore clinically significant. There are also chromosomal abnormalities, which although they do not have clinical significance for those affected, may carry an increased risk of diseases manifesting in their children. Chromosomal abnormalities are responsible for a large number of human genetic diseases. Patients with chromosomal abnormalities may present with different clinical features. These may include dysmorphic signs, developmental delays, mental retardation, organ malformations, skeletal malformations, and growth disorders varying in severity.

There are two types of chromosomal abnormalities: numerical abnormalities and structural abnormalities. In numerical abnormalities, the egg and sperm cells divide incorrectly resulting in an egg or sperm cell with too many or too few chromosomes. When the resulting cell combines with a normal egg or sperm cell, the resulting embryo has a chromosomal abnormality. A common type is trisomy in which the individual has three copies, instead of two, of a specific chromosome. Downs syndrome is an example of a trisomy with individuals generally having three copies of chromosome 21. In structural abnormalities, the chromosome structure is altered. This can take several forms including deletions, duplications, translocations, and inversions discussed in detail below. Other structural abnormalities include rings, in which a portion of the chromosome has broken off and formed a circle or ring, and isochromosome formed by the mirror image copy of a chromosome segment including the centromere. Chromosome instability syndromes are a group of disorders characterized by chromosomal instability and breakage. They often lead to an increased tendency to develop certain types of malignancies.

Alterations in chromosome number or structure can occur during development of an egg or sperm cell, or in the development of tumors. In addition, errors in cell division can also occur soon after fertilization, resulting in mosaicism, a condition in which the individual has cells with different genetic makeups. As an example, individuals with the mosaic form of Turner syndrome are missing an X chromosome in some, but not all of their cells. Chromosome abnormalities can be inherited from parents or be de novo. Accordingly, chromosome studies are often performed on parents when a child is found to have an abnormality. It should be noted that while embodiments of the present invention are well suited for use in humans, it is not intended to be limited as such; rather, it is anticipated that embodiments of the present invention have utility in identifying chromosomal abnormalities in all organisms.

Embodiments of the present invention relate to electronic karyotyping. As will be described below, electronic karyotyping provides a means for determining certain chromosomal abnormalities and variations in a manner that is relatively simple, quick, accurate, and well-suited for automation. Reference is made to the following definitions:

“Electronic karyotyping” is a molecular diagnostic tool that uses distinct patterns formed on chromosomal segments to detect genetic abnormalities. Electronic karyotyping leverages the same data used to create genome maps for sequence finishing efforts, thereby providing a relatively straightforward migration path from research into diagnostics.

A “chromosomal segment” or “chromosomal fragment” means a portion of a chromosome, or more specifically, a biomolecule having structural information that is to be determined using embodiments of the present invention. The chromosomal segment may be a biomolecule such as deoxyribonucleic acid, a ribonucleic acid, a protein, or a polypeptide. The chromosomal segment may be single-stranded or double-stranded. The terms “chromosomal segment” and “chromosomal fragment” may be used interchangeably herein.

A “probe” means a fragment of single-stranded DNA (ssDNA) or RNA, i.e., an oligonucleotide, which hybridizes to a complementary chromosomal segment. By detecting the presence of the probe and its location on the chromosomal segment, the location of complementary regions on that segment may be determined. Probes may be provided with a tag for the purpose of enhancing the ability of the probe to be detected in a nanodetector system.

A “marker” means a detectable moiety that can be bound to or otherwise located on a chromosomal segment at sequence specific locations. Markers may be selected that have sequence specificity for chromosomal segments of ssDNA and RNA, as well as doubled-stranded DNA (dsDNA). In one embodiment, markers may be oligonucleotide probes. In another embodiment, markers may be flaps formed at sequence-specific regions on dsDNA, as described in co-pending application U.S. Ser. No. 12/891,343, filed Sep. 27, 2010, incorporated herein by reference in its entirety. Alternatively, markers may comprise ternary complexes formed at sequence specific locations on dsDNA as described in U.S. Pat. No. 8,278,047, incorporated herein by reference in its entirety. As described above, like the subset probes, markers may be further provided with tags for the purpose of enhancing their ability to be detected in a nanodetector system.

It should be understood that embodiments of the present invention are not intended to be limited to any specific method for providing detectable regions on a chromosomal segment. Rather, it is intended to apply to the detection of any sequence specific probe or other marker hybridized, bound or otherwise present on a chromosomal segment. Further, in describing embodiments of the present invention, reference may be made to probes; however, unless otherwise noted, the description of the detection, characterization, location and velocity of probes is for purposes of simplicity only, and it is intended that the same methods may be applied to the detection, characterization, location and velocity of markers or any other detectable, sequence specific indicators on the chromosomal segment.

A “biomolecule analyte” is any molecule or assembly of molecules, e.g., a chromosomal segment having markers bound or otherwise located thereon, that is to be analyzed. An exemplary biomolecule analyte may include a single-stranded DNA or RNA chromosomal segment, with one or more sequence-specific oligonucleotide probes hybridized to a corresponding complementary portion of the chromosomal segment. A binding moiety may coat at least a portion of the single-stranded DNA or RNA chromosomal segment and/or the probes, and the resulting, coated structure is termed a biomolecule analyte as well.

A “tag” means a moiety that is attached to a marker to make the marker more visible to a nanodetector. Tags may be proteins, double-stranded DNA, single-stranded DNA, dendrimers, particles, or other molecules.

A “phenotype” means the composite of an organism's observable characteristics or traits, such as its morphology, development, biochemical or physiological properties, phenology, behavior, and products of behavior.

“Positional sequencing” means the determination of both the location and the identity of, for example, DNA sequences over long distances. Sequence specific markers can be chosen to query all size scales of DNA variation, from single nucleotides all the way to chromosomal. Using a single probe, genome-wide maps can be generated that can be used either for whole genome association studies or as scaffolds for assembly of short sequence reads obtained with traditional sequencing systems. Specific variants can be targeted for analysis, such as panels of SNPs or large-scale structural variants (e.g., translocations, rearrangements, amplifications, insertions, deletions). Unlike short read data produced by next-gen sequencing technologies that utilize sequencing-by-synthesis (SBS), positional sequencing provides detection of single molecules with many small segments of sequence that are ordered over distances that can range to as high as hundreds of kilobases. The location of each segment of sequence is captured along with its identity, greatly facilitating sequence assembly while dramatically reducing data burden.

A key to the approach described herein is the recognition that the electronic maps of probe or other marker positions generated by positional sequencing may be viewed as patterns that are directly analogous to the patterns of chromosomal staining used for karyotyping, yet with much higher resolution, and generated in a manner that is automated, rapid, relatively low cost, and completely objective.

Every chromosomal region has a distinct pattern of binding for any particular marker or combination of markers and a corresponding electronic profile that may be used in the same way large-scale structural variation is visualized with traditional karyotyping. However, when using the methods developed in connection with positional sequencing, karyotype data of much higher resolution may be generated. This is illustrated in FIG. 1. Specifically, FIG. 1 depicts, on the left 5, a conventional karyogram 15 of chromosome 2. Conventional karyotyping is made by staining the chromosome, or a segment thereof with a suitable dye such as Giemsa stain. In the case of human karyotyping, white blood cells are frequently used as a chromosomal source because they may be easily induced to divide and grow in tissue culture. The dye is typically applied after the cells have been arrested during cell division using a colchicine solution. Karyogram 15 shows the staining pattern in a small segment 25 of the chromosome. A distinctive pattern of staining can be seen in the karyogram 15, and variations of this pattern as compared to a reference are indicative of chromosomal abnormalities including deletions, duplications, translocations, and inversions at the studied segment. In the case of electronic karyotyping, shown on the right of FIG. 1, the method of which will be described in detail below, it can be seen that one may inspect even smaller segments 35 of the chromosome at much higher resolution. By detecting markers on the chromosome using electronic detection, a far more detailed pattern emerges as depicted by the electronic trace 45 which is a simulated data trace showing peaks indicative of marker locations on the chromosomal segment. This pattern is both more detailed than that obtainable using dyes and is machine-readable, thereby removing human subjectivity from interpretation of results. Variations in these distinctive patterns, as compared to a reference may be used in the same manner as conventional karyotyping to reveal and quantify chromosomal abnormalities including deletions, duplications, translocations, and inversions at higher resolution.

A methodology for analyzing a chromosomal segment, in accordance with an embodiment of the invention, may be carried out as follows. DNA representing an entire genome or a portion thereof, (i.e., a chromosomal segment), is isolated using any of the techniques widely known in the art. At least one sequence-specific marker set is provided. A marker is a detectable moiety that can be bound to or otherwise located on a chromosomal segment at sequence specific locations. Exemplary suitable markers include hybridizing oligonucleotides (i.e., probes having a known DNA sequence of a short length with a known composition). In one embodiment, the aims of the invention may be achieved using a single 6-mer oligonucleotide probe (or some other marker that has a sequence specific recognition capability based on a specific sequence of six nucleotides). If genomes were random in sequence, such a probe, on average, would bind every 4,096 nucleotides. The actual distribution may be significantly different from random, but nonetheless, 6-mer probes bind very frequently across the genome (about every 4 kb on average), and create unique patterns in any given chromosomal segment analyzed. Further, it is anticipated that by using more than one marker, high resolution electronic karyotyping may be achieved.

In one embodiment, each marker within the set is identical in length and sequence. Marker sets are selected such that each marker in the set is capable of binding to a specific sequence that appears one or more times in the chromosomal segment to be analyzed. In a preferred embodiment, the marker set comprises multiple copies of a single marker which binds at multiple locations within the chromosomal segment to be analyzed.

It is preferred that following the addition of markers to the chromosomal segment, the resulting complex is treated with a binding moiety. As will be described in detail below, the use of binding moieties increases both the diameter and persistence length of the complex, thereby enhancing its detection in a nanodetector. In a preferred embodiment, the binding moiety is a protein, and in a more preferred embodiment, it is the protein RecA.

The chromosomal segment, labeled with one or more marker sets, and optionally a binding moiety, i.e., the biomolecule analyte, is introduced into a device having a nanodetector capable of distinguishing differential electronic signals at locations on the chromosomal segment. The labeled segment is then translocated across the detector and changes indicative of i) no chromosomal segment present in the detector, ii) a chromosomal segment lacking a marker present in the detector, and iii) a chromosomal segment including a marker present in the detector are monitored. These electronic changes are recorded relative to their position on the chromosomal segment, thereby providing a distinct and characteristic pattern that is related to the positions of markers present on the chromosomal segment.

Raw data traces, in and of themselves, may not be particularly useful because they contain some level of uncertainty as to probe positions. However, the library of collected data may be combined to form a consensus map which provides, with high accuracy, the precise locations of the markers on the chromosomal segment being analyzed. The patterns on the resulting consensus map may be quantified and compared to reference patterns or patterns obtained from control and other test samples to reveal differences that are indicative of specific phenotypes in the origin of the chromosomal segment.

It should be noted that the pattern of marker positions need not be limited to those patterns produced by a single marker; rather, patterns characteristic of the positions of two or more markers are contemplated as well. It should further be noted that each set of markers, whether used alone or in combination with other marker sets, provides a unique and distinctive pattern. For example, if a marker set comprises, for example, 6-mer oligonucleotide probes having the sequence ATCGCG, this marker set has a distinct and characteristic pattern that differs from that produced by the 6-mer oligonucleotide probe CTTGTA. Thus, when comparing data to a reference, the pattern of the reference is preferably created using the same marker set (or sets) as was used to create the data being compared.

In the case where more than one marker is used to label the chromosomal segment of interest, the composition of the markers is chosen so as to reduce the chance of competitive binding to the chromosomal segment of interest.

Methods to increase the signal-to-noise ratio in nanopore or fluidic channel translocation of biomolecules that have been provided with markers are disclosed herein. In one embodiment, a single- or double-stranded biomolecule may be hybridized with a probe. The hybridized biomolecule may then be incubated with a protein or enzyme that binds to the biomolecule and forms at least a partial coating along the biomolecule.

RecA protein from E. coli typically binds single- or double-stranded DNA in a cooperative fashion to form filaments containing the DNA in a core and an external sheath of protein (McEntee, K.; Weinstock, G. M.; Lehman, I. R. Binding of the RecA Protein of Escherichia coli to Single- and Double-Stranded DNA. J. Biol. Chem. 1981, 256, 8835). DNA has a diameter of about 2 nm, while DNA coated with RecA has a diameter of about 10 nm. The persistence length of the DNA increases to around 950 nm, in contrast to 0.75 nm for single-stranded DNA or 50 nm for double-stranded DNA. T4 gene 32 protein is known to cooperatively bind single-stranded DNA (Alberts, B. M.; Frey, L. T4 Bacteriophage Gene 32: A Structural Protein in the Replication and Recombination of DNA. Nature, 1970, 227, 1313-1318). E. coli single-stranded binding protein binds single-stranded DNA in several forms depending on salt and magnesium concentrations (Lohman, T. M.; Ferrari, M. E. Escherichia Coli Single-Stranded DNA-Binding Protein: Multiple DNA-Binding Modes and Cooperativities. Ann. Rev. Biochem. 1994, 63, 527-570). The E. coli single-stranded binding protein may form a varied coating on the biomolecule. The f1 geneV protein is known to coat single-stranded DNA (Terwilliger, T. C. Gene V Protein Dimerization and Cooperativity of Binding of poly(dA). Biochemistry 1996, 35, 16652), as is human replication protein A (Kim, C.; Snyder, R. O.; Wold, M. S. Binding properties of replication protein A from human and yeast cells. Mol. Cell Biol. 1992, 12, 3050), Pf3 single-stranded binding protein (Powell, M. D.; Gray, D. M. Characterization of the Pf3 single-strand DNA binding protein by circular dichroism spectroscopy. Biochemistry 1993, 32, 12538), and adenovirus DNA binding protein (Tucker, P. A.; Tsernoglou, D.; Tucker, A. D.; Coenjaerts, F. E. J.; Leenders, H.; Vliet, P. C. Crystal structure of the adenovirus DNA binding protein reveals a hook-on model for cooperative DNA binding. EMBO J. 1994, 13, 2994). The protein-coated DNA is then translocated through a nanopore as has been demonstrated with RecA bound to double-stranded DNA (Smeets, R. M. M.; Kowalczyk, S. W.; Hall, A. R.; Dekker, N. H.; Dekker, C. Translocation of RecA-Coated Double-Stranded DNA through Solid-State Nanopores. Nano Lett. 2009). Translocation of protein bound to single-stranded DNA is contemplated herein. The protein coating functions in the same manner for single-stranded DNA and double-stranded DNA.

Coated biomolecule analytes typically have greater uniformity in their translocation rates, which leads to a decrease in positional error and thus greater accuracy.

As noted above, the marked chromosomal segment is introduced into an electronic detection device. The device may include a nanodetector. The nanodetector may include a nanopore or a fluidic channel detection system employing a nano-channel or micro-channel.

Mapping of analytes may be carried out using electrical detection methods employing nanopores, nano-channels or micro-channels using the methods described in U.S. patent application Ser. No. 12/789,817, filed May 28, 2010, the teachings of which are incorporated herein by reference in their entirety. It is contemplated that such methods may be applied to biomolecule analytes.

In one embodiment, current across a nanopore is measured during translocation of a DNA strand through the nanopore as shown in FIG. 2a. When used in embodiments of the present invention, a nanopore may have a diameter selected from a range of about 1 nm to about 1 μm. More preferably the nanopore has a diameter that is between about 2.3 nm and about 100 nm. Even more preferably the nanopore has a diameter that is between about 2.3 nm and about 50 nm. Changes in an electrical property across a nanopore may be monitored as the biomolecule analyte is translocated therethrough, with changes in the electrical property being indicative of the locations of markers on the analyte.

Specifically, for nanopore 100, a measurable current produced by electrodes 120, 122 runs parallel to the movement 110 of the biomolecule analyte 50, i.e., a DNA molecule 10 having a markers 20 bound thereto. Variations in current are a result of the relative diameter of the biomolecule analyte 50 as it passes through the nanopore 100. This relative increase in volume of the biomolecule analyte 50 passing through the nanopore 100 causes a temporary interruption or decrease in the current flow through the nanopore, resulting in a measurable current variation. Portions of the biomolecule analyte 50 including a marker 20 are larger in diameter than portions of the biomolecule analyte that do not include a marker. As a result, when the marker 20 passes through the nanopore 100, further interruptions or decreases in the current flow between electrodes 120, 122 occurs.

Changes in current flow are depicted in the waveform 200 in FIG. 2b. Analysis of the waveform 200 permits differentiation between regions of the biomolecule analyte that include markers and those that do not include markers, based, at least in part, on the detected changes in the electrical property. In FIG. 2b, the waveform 200 depicts the changes in a detected electrical property as the biomolecule analyte passes through the nanopore, and may be interpreted as follows.

Current measurement 210 represents a measured baseline current prior to passage of the biomolecule analyte through the nanopore from the cis side to the trans side. As the analyte enters the nanopore from the cis side of the nanopore, the current is partially interrupted forming a first trough 220 in the recorded current. When markers on the biomolecule analyte enter the nanopore, further decreases in current occur, causing a deeper, second troughs 230 in the current measurement. Upon passage of the markers entirely through the nanopore, a distal portion of the biomolecule analyte may remain in the nanopore. This causes the measured current 240 to rise to approximately the level of the first trough 220. Finally, once the entire biomolecule analyte has passed completely through the nanopore to the trans side, the measured current 250 returns to a level approximating that of the initial baseline level 210. Measurements of current variation are recorded as a function of time. While, for purposes of illustration, only two markers are shown in the Figure, in actual use, numerous markers would be present, and the detection and relative positions of these markers provides the chromosomal segment forming the base of the biomolecule analyte with a distinctive pattern. This pattern may be compared to a reference in order to identify chromosomal abnormalities.

In another embodiment, an electrical property such as electrical potential or current is measured during translocation of a biomolecule analyte through a nano-channel or micro-channel as shown in FIGS. 3 through 5, in which one embodiment of a fluidic channel apparatus is shown schematically.

In FIG. 3, the apparatus 300 comprises a fluidic micro-channel or nano-channel 302. The fluidic channel may be a micro-channel having a width selected from a range of about 1 μm to about 25 μm or a nano-channel having a width selected from a range of about 10 nm to about 1 μm. In the case of a micro-channel, the depth may be selected from a range of about 200 nm to about 5 μm, whereas in the case of a nano-channel, the depth may be selected from a range of about 10 nm to about 1 μm. In either case, the channel may have a length selected from a range of about 1 μm to about 10 cm.

A first pair of electromotive electrodes 304, 304′ is connected to a voltage source 306 and positioned in a spaced apart relationship at each end the channel. When a potential is applied to the electromotive electrodes, these electrodes provide an electrical current along the channel and may be used to provide or enhance an electromotive driving force 308 to a biomolecule analyte 50 in the channel. Other driving forces such as pressure or chemical gradients are contemplated as well. A second pair of electrodes 312, 312′, i.e., detector electrodes, is positioned preferably substantially perpendicular to the channel in a spaced apart relationship to define a detection volume 314. The second pair of detector electrodes 312, 312′ is connected to a detector 316, such as a voltmeter, which monitors an electrical property in the detection volume 314. In an embodiment where the detector 316 is a voltmeter, an electrical potential between the pair of detector electrodes 312, 312′, provided by the voltage source 306 and electromotive electrodes 304, 304′, is measured across the detection volume 314.

Additional relevant and applicable detection systems and methods are described in U.S. Pat. No. 8,246,799, U.S. Patent Publication No. 2010/009628, and U.S. Patent Publication No. 2010/0243449; these three publications are hereby incorporated by reference in their entireties.

The operation of the device is depicted schematically in FIGS. 4a-4e in which changes in an electrical property across a fluidic channel are monitored, as the biomolecule analyte is translocated therethrough. The changes in the electrical property are indicative of marked and unmarked regions regions of the biomolecule. In FIGS. 4a- 4e, the electromotive electrodes 304, 304′ and the current source 306 have been omitted for clarity.

In FIG. 4a, the fluidic channel 302 contains a biomolecule analyte 50 traveling therethrough. An electrical property, in this case electrical potential, is measured and recorded across the detection volume 314 by the detector electrodes 312, 312′ and the detector 316. The biomolecule analyte 50 is a chromosomal segment which has been provided with sequence specific markers using any of the methods described previously or otherwise known in the art. The analyte may be coated with a binding moiety, such as the protein RecA, to enhance detection.

Changes in electrical potential across detection volume 314 are depicted in the waveform 320 in FIG. 4a. Analysis of the waveform permits differentiation between regions of the biomolecule analyte that include markers and those that do not include markers, based, at least in part, on the detected changes in the electrical property. In FIG. 4a, the waveform depicts the changes in a detected electrical property as the biomolecule analyte enters the detection volume, and may be interpreted as follows.

Measurement 322 represents a measured baseline potential prior to passage of the biomolecule analyte through the detection volume. As the biomolecule analyte 50 enters the detection volume 314, it causes a change in the electrical property measured in the detection volume. This change causes a first trough 324 to be exhibited in the waveform 320.

FIG. 4b shows the device and waveform 320 once the portion of the biomolecule analyte 50 including a first marker has entered the detection volume 314. Entry of the first marker into the detection volume causes a further change in the electrical property measured in the detection volume. This further change causes a second trough 326 to be exhibited in the waveform 320. Once the first marker has exited the detection volume, the waveform returns to a level approximating that of the first trough 324.

FIG. 4c shows the device and waveform 320 once the portion of the biomolecule analyte 50 including a second marker has entered the detection volume 314. Entry of the second marker into the detection volume again causes a change in the electrical property measured in the detection volume. This change causes a third trough 328 to be exhibited in the waveform 320. This third trough 328 may approximate that of second trough 326.

FIG. 4d shows the device and waveform 320 once the portion of the biomolecule analyte 50 including a second marker has exited the detection volume 314, while an unmarked portion of the analyte is still present in the detector volume. The waveform 320 has returned to a level 330 approximating that of the first trough 324.

Finally, as shown in FIG. 4e, once the biomolecule analyte 50 has fully exited the detection volume 314, the waveform 320 has returned to a level 332 of the original baseline 322.

Analysis of the waveform 320 thus permits differentiation between portions biomolecule analyte which include markers and portions of the biomolecule analyte which do not include markers. Significantly, the relative locations of the markers produce a distinctive pattern which, when analyzed against a reference pattern can provide evidence of genetic abnormalities.

Embodiments of the present invention are well suited for the quantification and detection of certain abnormalities where the structure of the chromosome has been altered. These include deletions, duplications, translocations, and inversions of the chromosome as described in FIGS. 5-9.

FIG. 5 is a schematic representation of a chromosomal fragment 400, analyzed by the methods of the present invention, against a reference region 410 of a chromosome. In each of the fragment and the reference, the horizontal line 420 represents the ssDNA, dsDNA or RNA backbone of the reference or fragment, and the vertical tics 430 represent the relative locations of sequence specific markers located on the reference and the fragment. Dashed lines 440 represent correspondence between a marker on the fragment and a marker on the reference. As can be seen in FIG. 5, the markers on the fragment 400 have a one-to-one relationship with corresponding markers on the reference 410. Thus, if the reference is assumed to represent a normal section of a chromosome, it follows that the fragment has no abnormalities or structural variations.

FIG. 6 depicts a deletion event. The fragment of FIG. 6 has markers that correspond to markers on the left side of the reference, as well as markers that correspond to the right side of the reference. However, a portion 450 of the reference is missing. As such, the source organism for the fragment may have a phenotype corresponding to a chromosomal deletion. In humans, known chromosomal deletion disorders include Wolf-Hirschorn syndrome, caused by a partial deletion on the short arm of chromosome 4, and Jacobsen syndrome, also called the terminal 11q deletion disorder.

FIG. 7 depicts a duplication event. As can be seen, a portion 460 of the reference chromosome has been duplicated in the fragment, resulting in extra genetic material. In humans, chromosomal duplication is associated with Charcot-Marie-Tooth disease 1A which is believed to be caused by the duplication of the gene encoding peripheral myelin protein 22 (PMP22) on chromosome 17.

FIG. 8 depicts a translocation event. Translocations occur when a portion of one chromosome is transferred to another chromosome. There are two main forms of translocations. The first, called reciprocal translocation, occurs when segments from two different chromosomes have been exchanged. The second, known as Robertsonian translocation, occurs when an entire chromosome has attached to another at the centromere. These only occur with chromosomes 13, 14, 15, 21, and 22. An example of reciprocal translocation is depicted in FIG. 8, where it can be seen that the fragment has a left portion that corresponds to a location on reference chromosome 9, and a right portion that corresponds to a location on reference chromosome 22. In humans, chromosomal translocation is associated with schizophrenia, as well as several varieties of cancer, leukemia, lymphoma, sarcoma, and other disorders.

FIG. 9 depicts an inversion event. Inversions occur when a segment of a chromosome is reversed end to end, i.e., when a single chromosome undergoes breakage and rearrangement within itself. Inversions are of two types: paracentric and pericentric. Paracentric inversions do not include the centromere and both breaks occur in one arm of the chromosome. Pericentric inversions include the centromere and there is a break point in each arm. In FIG. 9, a first portion 470 of the fragment can be seen to properly correspond to the reference, while a second portion 480 of the fragment is seen to be inverted.

Inversions usually do not cause abnormalities in carriers as long as the rearrangement is balanced with no extra or missing DNA. However, in individuals that are heterozygous for an inversion, there is an increased production of abnormal chromatids. Lowered fertility results due to production of unbalanced gametes. The most common inversion seen in humans is on chromosome 9, at inv(9)(p12q13). This inversion is generally considered to have no deleterious or harmful effects, but there is some suspicion it could lead to an increased risk for miscarriage or infertility for some affected individuals.

A comprehensive description of human chromosomal abnormalities can be found in ISCN 2013; An International System for Human Cytogenetic Nomenclature (2013), Ed. Shaffer, L.G., et al.

Employing the methods described herein, it is possible to detect marker binding positions on chromosomal segments in a manner that produces highly detailed patterns of marker positions. When compared to a known reference, these patterns can provide indications of a wide variety of genetic abnormalities and structural variations.

### EXAMPLE

A data trace of a segment of DNA from the yeast Saccharomyces cerevisiae obtained using the methods of the present invention is shown in FIG. 10a. In that Figure, an ssDNA fragment having a length of between about 100 kb and 150 kb was marked using a sequence-specific probe, coated with RecA protein, and translocated through the detection volume of a fluidic nano-channel-based nanodetector. Electrical signals indicative of i) no DNA 500 in the detection volume, i.e. baseline, ii) unmarked DNA 510 in the detection volume, and iii) sequence specific markers on the DNA 520 in the detection volume, were monitored and recorded. These signals can be translated into a pattern 530, shown in FIG. 10b, which is characteristic of that particular DNA segment. By comparing the pattern 530 of FIG. 10b to a pattern on a reference portion of the same Saccharomyces cerevisiae DNA segment to detect differences, abnormalities and structural variations in the studied segment can be detected.

### Equivalents

Those skilled in the art will readily appreciate that all parameters listed herein are meant to be exemplary and actual parameters depend upon the specific application for which the methods and materials of embodiments of the present invention are used. It is, therefore, to be understood that the foregoing embodiments are presented by way of example only and that, within the scope of the appended claims and equivalents thereto, the invention may be practiced otherwise than as specifically described.

The described embodiments of the invention are intended to be merely exemplary and numerous variations and modifications will be apparent to those skilled in the art. All such variations and modifications are intended to be within the scope of the present invention as defined in the appended claims.

What is claimed is:

1. A method for analyzing chromosomal segments, the method comprising comprises the steps of:
a) providing a chromosomal segment to be analyzed,
b) providing at least one sequence-specific marker set, each marker within the set binding or otherwise locating at a sequence-specific location on the segment to be analyzed,
c) allowing the one or more marker sets to bind or otherwise locate on the chromosomal segment to form an analyte,
d) introducing the analyte into a device comprising a nanodetector;
e) translocating the analyte through the nanodetector,
f) monitoring changes in an electrical property as the analyte translocates through the nanodetector, such changes indicative of at least one of i) no chromosomal segment present in the nanodetector, ii) a portion of the chromosomal segment lacking a marker present in the nanodetector, or iii) a portion of the chromosomal segment having a marker present in the nanodetector, and
g) recording the changes in the electrical property relative to their positions on the analyte to form a pattern distinctive of the chromosomal segment.

2. The method of claim 1, further comprising coating at least a portion of the analyte with a binding moiety prior to introducing the analyte into the device.

3. The method of claim 2, wherein the binding moiety comprises one or more proteins.

4. The method of claim 3, wherein the one or more proteins comprises at least one member selected from the group consisting of RecA, T4 gene 32 protein, f1 geneV protein, human replication protein A, Pf3 single-stranded binding protein, adenovirus DNA binding protein, and E. coli single-stranded binding protein.

5. The method of claim 1, wherein the nanodetector comprises a fluidic nanopore.

6. The method of claim 1, wherein the nanodetector comprises a fluidic micro-channel or nano-channel.

7. The method of claim 1, wherein the marker comprises an oligonucleotide probe.

8. The method of claim 1, wherein the marker is provided with a tag.

9. The method of claim 1, which comprises the additional step of comparing the pattern distinctive of the chromosomal segment to an analogous pattern on a reference chromosome to identify differences between the reference chromosome and the analyte.

10. The method of claim 1, further comprising quantifying the pattern distinctive of the chromosomal segment.

11. A method for analyzing structural variation in genomes, the method comprising the steps of:
a) isolating DNA representing an entire genome or a portion thereof to be analyzed;
b) providing at least one sequence-specific marker set, with each marker in the set capable of binding to or otherwise locating at a specific sequence that appears one or more times in the isolated DNA;
c) allowing the one or more marker sets to bind or otherwise locate on the isolated DNA to form an analyte;
d) introducing the analyte into a device comprising a nanodetector having a detection volume;
e) translocating the analyte through the detection volume of the nanodetector,
f) monitoring changes in an electrical property as the analyte translocates through a detection volume in the nanodetector, such changes indicative of i) no DNA segment present in the detection volume, ii) a DNA segment lacking a marker present in the detection volume, and iii) a DNA segment having a marker present in the detection volume;
g) recording the changes in the electrical property relative to their positions on the analyte, thereby revealing a pattern of changes in the electrical signal that is related to the positions at which markers are present on the isolated DNA; and
h) comparing the pattern to analogous patterns from control and test samples to reveal differences that are indicative of specific abnormalities or structural variations in the test sample.

12. The method of claim 11, further comprising coating at least a portion of the analyte.

13. The method of claim 12, wherein the binding moiety comprises one or more proteins.

14. The method of claim 13, wherein the one or more proteins comprises at least one member selected from the group consisting of RecA, T4 gene 32 protein, f1 geneV protein, human replication protein A, Pf3 single-stranded binding protein, adenovirus DNA binding protein, and E. coli single-stranded binding protein.

15. The method of claim 11, wherein the nanodetector comprises a fluidic nanopore.

16. The method of claim 11, wherein the nanodetector comprises a fluidic micro-channel or nano-channel.

17. The method of claim 11, wherein the marker comprises an oligonucleotide probe.

18. The method of claim 11, wherein the marker is provided with a tag.

19. The method of claim 11, further comprising quantifying the pattern recorded in step g).
